(19) United States
(12) Patent Application Publication
(10) Pub. No.: US 2022/0217833 A1
(43) Pub. Date: Jul. 7, 2022

(54) PLASMA SURFACE SANITIZER AND ASSOCIATED METHOD

(71) Applicant: ALPHATECH INTERNATIONAL LIMITED, Hong Kong (CN)

(72) Inventor: Herman Yik Wai TSUI, Hong Kong (CN)

(73) Assignee: ALPHATECH INTERNATIONAL LIMITED, Hong Kong (CN)

(21) Appl. No.: 17/608,990

(22) PCT Filed: May 5, 2020
(86) PCT No.: PCT/CN2020/088573

§ 371 Date: Nov. 5, 2021

Related U.S. Application Data
(60) Provisional application No. 62/843,531, filed on May 5, 2019.

(51) Int. Cl. H05H 1/24; A61L 2/14

(52) U.S. Cl. CPC H05H 1/2431; A61L 2202/11; A61L 2/14

(57) ABSTRACT

The plasma rail device of the invention is a novel method and device to generate plasma right at the surface or slight above the surface for surface treatment, specifically for surface sanitization. The device generally comprises pairs of electrodes held together by a frame. The device features a fame that holds and positions the electrodes in a close proximity towards the surface under treatment. Discharges are created in the space bounded by the electrodes to generate plasma on the surface and above the surface for treatment so that the device can be positioned outside the object under treatment.

### FIELD OF THE INVENTION

The present invention relates generally to an application of cold plasma for surface treatment and disinfection and more particularly pertains to a plasma device for surface disinfection. It further relates to a method for generating controllable and uniform plasma directly to the treatment surface to create desirable surface properties, including disinfection.

### BACKGROUND OF THE INVENTION

Cross-contamination and cross-infection can take place over air transmission or through commonly touched surfaces. Material surfaces can harbor germs. Some bacterial species, for example, methicillin-resistant Staphylococcus aureus (MRSA), can survive for 4 to 5 months or more on dry surfaces, and viruses, such as norovirus, can survive for up to one week.

Plasma technology has been proved to be very effective in air sanitization in recent years (see for example, U.S. Pat. No. 8,361,402 B2, Apparatus for Air Purification and Disinfection, Tsui). Plasma is referred to as the 4ₜₕ state of matter, and is a partially ionized gas composed of freely moving ions, electrons, and neutral particles. While overall plasma is electrically neutral, it is electrically conductive. This property allows the injection of electrical energy into the space occupied by the plasma. Depending on the operating conditions, plasma can consist of charged particles (electrons and ions), excited species, free radicals, ozone and UV photons, which are capable of decomposing chemical compounds and destroying microbes. The energy of the electrons can be utilized for exciting atoms and molecules, thereby initiating chemical reactions and/or emission of radiations. These emissions, particularly in the UV spectral region, can initiate photo-physical and photo-chemical process by breaking molecular bonds. The energetic electrons are able to induce the breakdown of some chemical bonds of the molecules, collide with the background molecules resulting in the breakdown of molecular chain, ionization and excitation, and generation of free atoms and radicals such as O, OH or HO₂. The radicals can attack hazardous organic molecules and are useful in decomposing pollutants in air. The disassociation of O₂ provides the required O to combine with O₂ to form ozone. The low energy electrons can attach to neutral atoms or molecules to form negative ions, which can enhance reactions in decomposing pollutants and destruction of microbes. Furthermore, to be effective, a delivery mechanism is usually necessary to deliver the charged particles and the active species to the surface to be treated.

Plasma can be created by electrical means in the form of gaseous discharges whereby a high voltage is applied to a set of electrodes, the anode and the cathode. When the applied voltage is sufficiently high and becomes greater than the breakdown voltage, arcs begin to develop across the anode and the cathode electrodes. Often, the electrodes suffer from degradation and overheating difficulties during prolong usage.

A number of approaches for surface sanitization with plasma have been suggested, some of which deals with the ways for delivering charged particles and other active species from a plasma generation device to the treatment surface. For examples,

Patent Application Publication U.S. 20040005261 A1 (Plasma Sterilization Apparatus, Ko) describes the sterilization of articles in a vacuum chamber by drawing in plasma and active species generated in another chamber.

U.S. Pat, No. 7,633,231 B2 (Harmonic Cold Plasma Device And Associated Methods, Watson) describes the use of a plasma gun device with helium gas injection and magnetic system to deliver the plasma and active species to the surfaces to be treated.

U.S. Pat. No. 9,236,227 B2 (Cold Plasma Treatment Devices and Associated Methods, Watson et al.) describes the use of a plasma gun device to deliver the highly charged ions and reactive species to the patient to inhale.

U.S. Pat. No. 9,623,132 B2 (Plasma-generated Gas Sterilization Method, Krohmann et al.) describes the generating a plasma from air to produce reactive nitrogen and oxygen species, which are brought in contact with water to form a mixture and then directed to the objects for sterilization.

Patent EP 2052097 B1 (Plasma Surface Treatment Using Dielectric Barrier Discharges, Boulos et al.) describes surface coating treatment with the use of a plasma torch having coating material feeding the plasma torch.

In another known approach, the object to be treated is placed close to the location where the plasma generating electrodes are positioned. Typically, the electrodes are arranged in a sandwiched or woven configuration, as exemplified in the following references.

Patent Application Publication U.S. 20120039747 A1 (Treating Device for Treating a Body Part of a Patient with a Non-thermal Plasma, Morfill et al.) disclosed a plasma generating electrode configuration with a dielectric insulator sandwiched between two electrodes, one of which is in the form of a plate and another is a wire mesh. Surface discharges occur on the surface of the dielectric insulator in the void space of the wire mesh. A similar sandwich electrode configuration is also disclosed in Patent Application Publication US 20180206321 A1 (Electrode Assembly and Plasma Source for Generating a Non-Thermal Plasma, and Method for Operating a Plasma Source, Morfill et al.) with additional dielectric insulators covering the outermost (top and bottom) of the electrodes, forming a 5-layer structure.

Patent Application Publication U.S. 20120039747 A1 also disclosed another configuration comprising of one set of electrodes being insulated and another set being bare or insulated. This configuration is also disclosed in U.S. Pat. No. 7,037,468 B2 (Decontamination of Fluids or Objects contaminated with Chemical or Biological Agents Using a Distributed Plasma Reactor, Hammerstrom et al.) and Patent Application Publication U.S. 2005/0249646 A1 (Gas Treatment Apparatus, Iwama et al.). In this configuration, plasma generating discharges occur at the intersection of the crossing electrodes.

Still in another known approach, the object to be treated is placed in between the high voltage electrodes and the ground electrodes, as shown in the following example.

U.S. Pat. No. 9,295,280 B2 (Method and Apparatus for Cold Plasma Food Contact Surface Sanitation, Jacofsky et al.) describes the use of plasma for surface sanitation where the treated surface acts a as the ground electrode or a grounding rod assembly is placed on the underside of the surface to be treated (i.e., the surface to be treated is sandwiched between the high voltage electrode and the ground electrode).

These prior approaches require additional mechanisms to deliver charged particles and other active species to the surfaces to be treated or by sandwiching the object to be treated in between the high voltage and ground electrodes or by having plasma generating discharges restricted to the surface of the dielectric and at the intersection of the electrode pair. Therefore, it is desirable to develop a method and a device that can easily generate plasma directly on or above the surface to be sanitized without the need for special delivery or configurational arrangement, tangling the treating surfaces with the device.

### SUMMARY OF THE INVENTION

In view of the aforesaid disadvantages present in the prior art and based on the principles as mentioned above, the method and device of the present invention provides a process of generating plasma directly on or above the surface to be sanitized. This object is achieved with the use of a plasma trail device.

The plasma rail device embodies a novel method and device design to generate plasma right at the surface or slightly above the surface for surface treatment, specifically for surface sanitization. The device generally comprises pairs of high voltage electrodes and ground electrodes arranged in alternate polarity, i.e., alternate high voltage and ground electrodes, and powered by an alternating current power source. The high voltage electrodes are covered by an insulated dielectric. The ground electrodes can be bared or covered by an insulated dielectric. The pairs of electrodes are powered by an alternating current power source with the high voltage electrodes connected to the high voltage end of the power source and the ground electrodes connected to the low voltage end of the power source. Discharges is created in the space between the pairs of electrodes and on the surface of the object to be treated.

In a first preferred aspect, there is provided a system for surface treatment and sanitization, comprising:

(a) at least one pair of electrodes, a high voltage electrode and a ground electrode, with the high voltage electrode being covered by a dielectric material to provide electrical insulation and the ground electrode being either bare or covered by a dielectric material;

(b) a frame that holds and positions the electrodes in a predefined, and preferably adjustable, distance towards the surface to be treated; and

(c) a power supply for supplying high voltage alternating current to the electrodes; whereby the electrodes generate plasma in the space separating the electrodes and on the surface of the object being treated.

The power supply may be adjustable to adjust the amplitude, waveform period and shape of the voltage applied to the electrodes so as to maximize plasma activity and minimize the generation of unwanted bi-product gases.

Insulators of the electrodes may be in the form of a dielectric tube made of glass or plates.

Conductors of the electrodes may be made of conducting sheets, mesh or deposits.

The voltage supplied may be in a range of 10 kilovolts to 50 kilovolts.

The waveform period may be in a range of 10⁻¹ ms to 10² ms

The separation between a pair of electrodes is preferably in a range of 1 mm to about 20 mm. The distance between the electrodes and the surface to be treated is preferably in the range between 0 mm to 20 mm.

The method may further comprise adjusting the amplitude, waveform period and shape of the voltage applied to the electrodes to maximize plasma activity and minimize the generation of unwanted bi-product gases.

The device of the present invention has a high-voltage alternating current power source for controlling the amplitude, waveform period and shape of the voltage applied to the electrodes and hence the operation with plasma discharges of selected conditions. The high-voltage alternating current power source may be a high-voltage generator. The amplitude, waveform period and shape of the voltage applied to the electrodes may be adjusted according to the desired treatment strength and treatment time in the plurality of reactors. The system generally comprises of a plurality of reactors arranged in alternate high voltage and ground electrodes allowing the configuration and overall size be designed to result in a suitable treatment strength and time.

The high voltage electrodes are covered by an insulator. The ground electrodes can be bared or covered by an insulator. The insulated electrodes include insulators which may be in the form of dielectric tubes or plates.

The system may further include a blower unit for driving air over the object to be treated to reduce heating of the object surface.

It is an advantage of discharges generation on the surface of the object to be treated to create plasma for directly treating the surface.

It is another advantage of discharges generation right above of the surface of the object to be treated to create plasma for directly treating the surface.

An even further advantage of at least one embodiment of the present invention is to provide a method and device for generating plasma directly on or above the surface of the object to be treated (including sanitized) without the need for delivering the charged ions and reactive species or configurational arrangement, thus overcoming the disadvantages of the prior art.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF THE DRAWINGS

Specific embodiments of the invention will now be described by way of example with reference to the accompanying drawings wherein:

FIG. 1 illustrates the components of a surface treatment device of the present invention;

FIG. 2 illustrates the electrode and the frame assembly according to a preferred embodiment;

FIG. 3 illustrates the electrode construct according to a preferred embodiment;

FIG. 4 illustrates the electrode assembly according to an another embodiment;

FIG. 5 illustrates an another embodiment of the electrode assembly with insulated electrodes placed within the circular cut-outs of the ground plate;

FIG. 6 illustrates an another embodiment of the electrode assembly with insulated electrodes placed within the hexagon cut-outs of the ground plate;

FIG. 7a, 7b illustrate an another embodiment with adjustable electrode positions;

FIG. 8 shows a prototype device constructed according to the present invention to demonstrate the effects of surface treatment;

FIG. 9 shows the experiment results using the prototype of FIG. 8

### DETAILED DESCRIPTION OF THE INVENTION WITH EMBODIMENTS

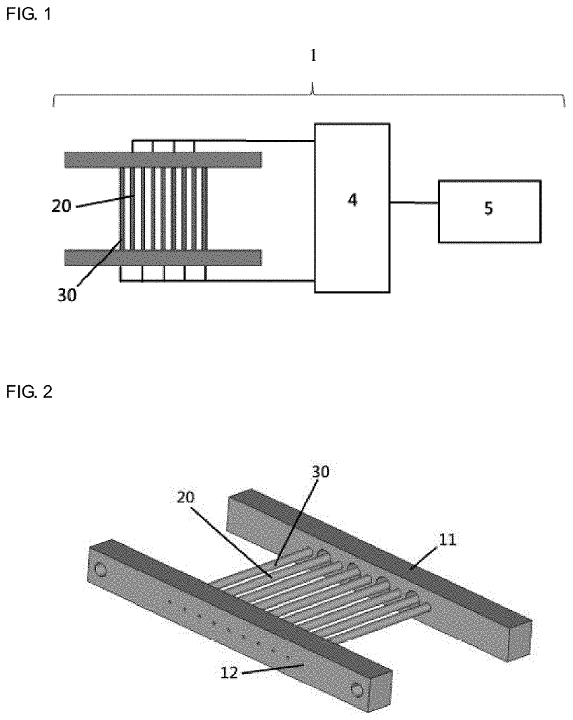

Reference will now be made in detail to a preferred embodiment of the invention. Referring now to the drawings, FIG. 1 generally shows system components of a surface treatment system 1 comprising the electrode assembly 10 with the high voltage electrodes 20, the low voltage electrodes 30 and its associated power supply 4 and controller 5. The power supply and controller create and sustain discharges with specific plasma parameters predetermined and controlled by the high-voltage alternating current power source. As illustrated in the FIG. 1, the electrodes 20, 30 may be connected to a high-voltage alternating current power supply 4 having an electronic control unit 5. The power supply 4 can provide sufficient voltage to cause breakdown and to generate plasma directly on or above the surface of the object to be treated (including sanitized) without the need for delivering the charged ions and reactive species. The voltage applied to the electrodes 20, 30 may be controlled within a range of 10 kilovolts to 50 kilovolts. The waveform period may be controlled within a range of 10₋₁ ms to 10₂ ms.

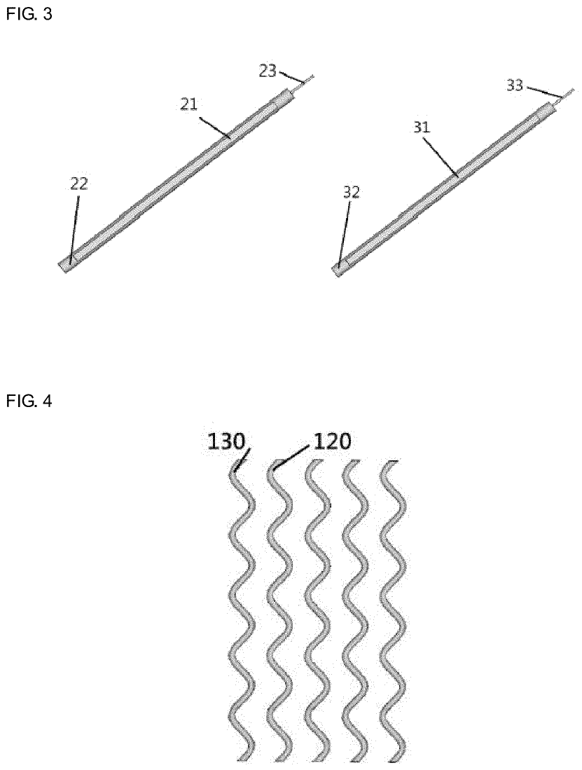

FIG. 2 shows a preferred embodiment of the electrode assembly 10 comprising the high voltage electrodes 20, the low voltage electrodes 30. The electrodes are held in place by the holders 11 and 12. Besides the planar form, the assembly can take on other forms, such as a cylinder or sphere. As illustrated in FIG. 3, the high voltage electrode 20 has a conductor 21 covered by an insulator 22 and a wire connection 23 to the power supply. The low voltage electrode 30 has a conductor 31 which can be bare or covered by an insulator 32, and a wire connection 33 to the power supply. The insulators can be made of dielectric materials such as glass or ceramic in the form a cylindrical tube as in this preferred embodiment. They may also be in the form of plates or made from any insulating or dielectric material. The insulators can also be in the form of a dielectric coating. The electrode conductors 21, 31 of the electrodes 21, 31 may be made of conducting sheets, mesh or deposits. The distance between a pair of electrodes 20, 30 may be in the range of about 1 mm to about 20 mm. Electrical discharges are created in the space bounded by the electrodes to generate plasma on the surface and above the surface for treatment.

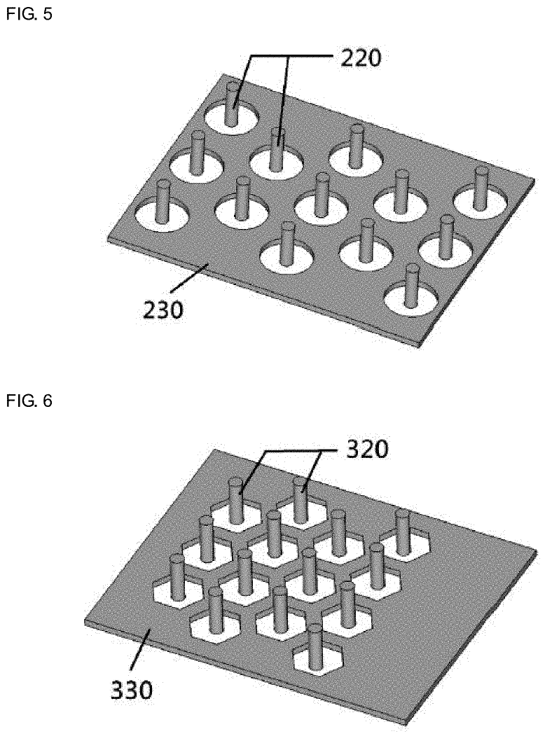

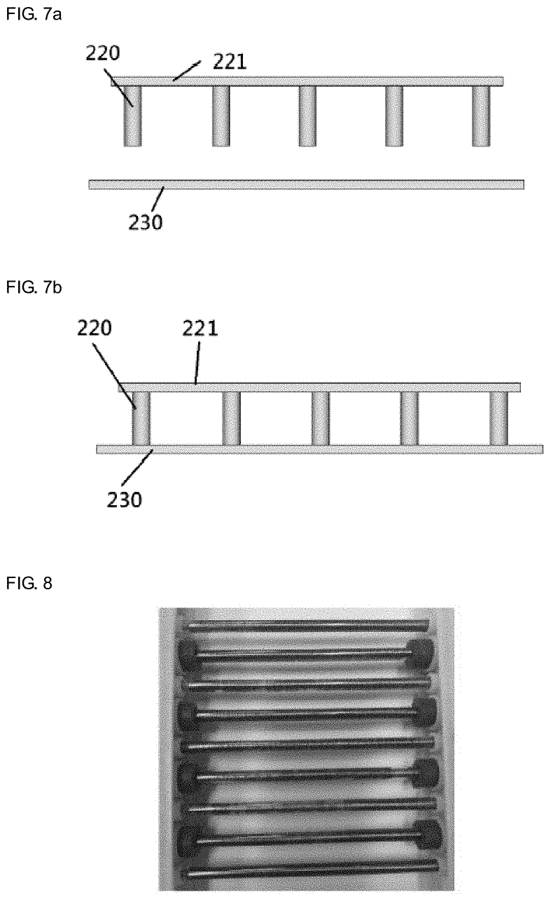

The electrodes can take on other shapes, for example, in the wavy shaped the voltage electrodes 120, the low voltage electrodes 130 as shown in FIG. 4. The electrode assembly is not limited to the form of a rail. In the embodiment shown in FIG. 5, the ground electrode 230 is a conducting sheet with circular cut-outs to accommodate the insulated high-voltage electrodes 220. FIG. 6 shows another embodiment with the insulated high-voltage electrodes 320 placed in the hexagon cut-outs of the ground electrode plate 330. Although the embodiments are shown in the planar form, the assembly can take on other forms, such as a cylinder or sphere. As an additional feature illustrated in FIGS. 7a and 7b, the high voltage electrodes 220 mounted on a support 221 can be recessed behind the ground electrode plate 230 when not in use as shown in FIG. 7a and can be moved into the operating position when they are used for surface treatment and sanitization as shown in FIG. 7b. An alternate embodiment is to have the ground electrode plate 230 moved to become flushed with the high-voltage electrodes during surface treatment usage.

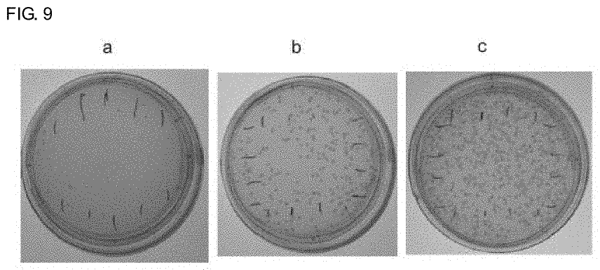

The superior surface sanitization performance of a prototype device of this invention (see FIG. 8) than a prototype device according to a prior art is confirmed in a comparison test. In this test, the petri dishes were pre-loaded with bacteria, treated by the corresponding devices operating at comparable plasma conditions. In the photos of FIG. 9, each ‘dot’ on the petri dish represents a colony of bacteria. FIG. 9c is the ‘control’ in which the petri dish has not been treated by any of the devices, showing the initial concentration of the bacteria. FIG. 9a shows the outcome of a petri dish treated by the device of this invention (device shown in FIG. 8). FIG. 9b shows the outcome of a petri dish treated by a device according to a prior art. There are much fewer colonies of bacteria in FIG. 9a than in FIG. 9b, indicating the device of this invention is more effective in surface sanitization.

The electrode and frame assembly (for example according to a preferred embodiment in FIG. 2) can be applied to a smooth surface with the surface of the electrodes (20, 30) either resting on the surface to be treated or above the surface to be treated with a distance not larger than the separation between the electrodes (20, 30). In the preferred embodiment shown in FIG. 2, the separation between the electrodes (20, 30) may be in the range from 1 mm to 20 mm. The distance between the electrode surface and the surface to be treated may be in the range of 0 mm to 20 mm. Typical treatment time is a fraction of a second to a few seconds. By moving air (for example using a fan) though the gap space between the electrode surface and the surface to be treated, the device of this invention can simultaneously sanitize air.

In an alternative embodiment as shown in FIG. 5, the high voltage electrodes 220 can be mounted on a movable support 221 to optimize the distance between the electrode tips and the surface to be treated.

The surface treatment is not limited to a surface of size smaller than the electrode assembly. The electrode assembly can be used to treat a large surface by sliding or moving the electrode assembly over the surface to be treated. Alternatively, the surface to be treated can be moved under the electrode assembly, for example, the electrode assembly can be positioned above the rubber handrail of an escalator which moves continuously underneath the electrode assembly. As the electrodes are positioned above the moving surface, thereby avoiding physical contact and eliminate mechanical wear and tear.

With a treatment time of seconds to sanitize a surface under the electrode assembly, an electrode assembly of the size of a small notebook computer can effectively disinfection the surface of a desk within minutes. By attaching the electrode assembly to a mobile device (a robot or a drone), a mobile electrode assembly can sanitize surface of a room in minutes. By allowing air to flow (for example, via the movement of the mobile device or by using a fan) through the gap space between the electrode surface and the surface to be treated, the device can simultaneously sanitize air.

In the case of a curved surface, the frame holding the electrodes (11, 12 in FIG. 2) may be made in a curved shape to match the curvature of the surface to be treated. Furthermore, the holder of the preferred embodiment (11, 12 in FIG. 2) may be made of flexible materials or be constructed in the form of a flexible chain to enable the electrodes to conform to an arbitrarily curved surface. For the alternative embodiment as shown in FIG. 5, the high voltage electrodes 220 can be mounted on a flexible support 221 together with a flexible ground electrode 230 to enable the electrode tips to conform to an arbitrarily curved surface and an optimal distance between the electrode tips and the surface to be treated.

In this invention, the distance between the electrode surface and the surface to be treated is not required to be fixed but can be varied within a reasonably range of 0 mm to 20 mm. The device is therefore able to sanitize not only smooth surface but also non-flat surface with surface irregularities up to 20 mm.

For the device of this invention, the construct and arrangement of the electrodes allow the sanitizer to be easily applied above the surface of the object to be treated. In comparison with the prior art devices, the device of the present invention is simple to construction and yet is flexible and more effective in terms of treating surfaces. There is no specific requirement to ground the surface of the object, i.e., the object to be treated effectively become part of the electrical circuit. For the frame, the basic requirement is that it can hold and position the electrodes in a required close distance towards the treatment surface. So long the frame can accomplish such requirement, it can be constructed in various way in various shape and material. For example, it may be made a flexible material for treating curved or irregular surfaces. It may also embed a fan mechanism for directing the air to the treatment surface. It may also have wheel or sliding mechanism for facilitating moving over the surface under treatment. This invention allows application of plasma treatment to various objects, for example, treating the elevator button panel and other construction surfaces in common areas to reduce virus and bacterial infection and transmission in community. This is because, unlike prior art devices, there is also no requirement to put the object in between the electrodes. Beside the uniqueness of the invention to overcome some of the deployment complexity of prior arts, the device of this invention is able to sanitize a surface better than the device of prior arts.

It is to be understood that the phraseology and terminology employed herein are for the purpose of description and should not be regarded as limiting. While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims. It is further under- stood that the present invention can be practiced even without referring to these specific examples because the essence of the present invention does not lie in technical difficulty or complexity but in the novel ideas itself. Once the idea is known, the practice of it is within ordinary skill in the art.

1. A device for treating surfaces, comprising:
(a) at least one pair of electrodes, separated by a distance between 1-20 mm and capable of generating plasma;
(b) a frame holding and positioning the electrodes towards a surface under treatment in a distance between 0-20 mm;
(c) a power supply for supplying high voltage alternating current to the electrodes; and
(d) a controller controlling generation of the plasma and treatment process.

2. The device according to claim 1, wherein one electrode is of a higher voltage and is insulated with a dielectric material and another electrode is of a lower voltage and insulated or bare.

3. The device according to claim 1, wherein one electrode has a conductor in the form of conducting sheets, mesh, wire or deposits.

4. The device according to claim 1, wherein the frame has a flat surface which is parallel to an plan formed by the electrodes and has a vertical distance towards the plan between 0 mm to 20 mm, which determines an distance between the electrodes and a surface under treatment.

5. The device according to claim 1, wherein the frame has a curved surface which conforms to a surface under treatment.

6. The device according to claim 1, wherein the frame has a flexible surface which can change shape to suit to a surface under treatment.

7. The device according to claim 1, wherein the frame housing a fan or fanning device to direct air towards a surface under treatment.

8. The device according to claim 1, wherein the frame has a sliding surface to facilitate movement of the device on a surface under treatment.

9. The device according to claim 1, wherein the frame has a plurality of wheels to facilitate movement of the device over a surface under treatment.

10. The device according to claim 1, wherein the power supply can supply electricity of a voltage between 10 kv and 50 kv.

11. The device according to claim 1, wherein the controller can adjust the power supply and control amplitude, waveform period and shape of a voltage applied to the electrodes so as to maximize plasma treatment and minimize generation of unwanted bi-product gases.

12. The device according to claim 11, wherein the waveform period is in a range of 10⁻¹ ms to 10² ms.
